(19) United States
(12) Patent Application Publication
(10) Pub. No.: US 2002/0160512 A1
(43) Pub. Date: Oct. 31, 2002

(54) Ex vivo expansion of mammalian hematopoietic stem cells

(76) Inventor: Chu-Chih Shih, Duarte, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/134,516

(22) Filed: Apr. 30, 2002

Related U.S. Application Data
(63) Continuation of application No. 09/570,055, filed on May 12, 2000, now abandoned.
(60) Provisional application No. 60/134,131, filed on May 14, 1999.

(51) Int. Cl. C12N 5/06; C12N 5/08

(52) U.S. Cl. 435/372; 435/354

(57) ABSTRACT

A method for the ex-vivo expansion of mammalian hematopoietic stem cells (HSC) comprises culturing isolated HSC in a culture system comprising a culture medium in the presence of a stem cell expansion promoting factor (SCEPF) which may be obtained by culturing murine stromal cells in the presence of an amount of leukemia inhibitory factor (LIF) sufficient to stimulate the production and secretion of said expansion promoting factor.

This application is a continuation of U.S. application Ser. No. 09/570,055, filed May 12, 2000, which claims priority from provisional application No. 60/134,131, filed May 14, 1999.

### FIELD OF THE INVENTION

The present invention is in the field of ex vivo maintenance and expansion of stem cell populations for regeneration in recipient patients.

### BACKGROUND OF THE INVENTION

During the last 20 years, hematopoietic stem cell transplantation (HSCT) has been conclusively proven to provide definitive therapy for a variety of malignant and non-malignant hematological diseases and myelopoietic support for patients undergoing high-dose chemotherapy. However, the use of HSCT in clinical therapy is limited. Limitations include a lack of sufficient donors, the need for either bone marrow (BM) harvest or pheresis procedures, the occurrence of a period of BM aplasia leading to severe, prolonged neutropenia and thrombocytopenia, and the potential for tumor contamination in autologous stem cell transplantation. This has resulted in an interest in the development of expansion strategies for human hematopoietic stem cells (HSC) in vitro to overcome some of these limitations.

The ex vivo HSC generated by such expansion strategies could support multiple cycles of chemotherapy. In addition, they would also allow for transplantation of HSC to patients who are without matched donors. An ex vivo expansion method also would provide for a tumor free product and facilitate the transduction of vectors into HSC for gene therapy. The extended neutropenia and thrombocytopenia may be abrogated by expanded cells from umbilical cord blood.

The development of ex vivo culture conditions that facilitate in vitro maintenance and expansion of long-term transplantable HSC is a crucial component and major challenge in stem cell research. This is a necessary first step towards a better understanding of the regulatory process that governs the development of all hematopoietic lineages from HSC. Several studies have shown that in ex-vivo culturing of HSC, with or without stromal cells, the HSC cells will retain the capability to engraft human recipients. However, it is not known from these studies whether the number of transplantable HSC has changed. Furthermore, all of these studies have involved autologous transplantation, making it difficult to determine whether the repopulation is derived from the surviving endogenous stem cells or from the engrafted cells. It is controversial whether primitive human hematopoietic cells are actually expanded during ex-vivo culture, because different assays and culture conditions have been used in different studies. Human hematopoietic stem cells from highly purified subfractions of CD34⁺ cells possess the greatest proliferative potential resulting in large expansion of colony-forming cells (CFC), while long-term culture initiating cells (LTC-IC) show either a slight reduction or a moderate increase. HSC are defined as having both the capability of self-renewal and the ability to differentiate into at least eight distinct hematopoietic cell lineages. Hematopoietic progenitors in human bone marrow can be identified by the expression of the CD34 antigen. Enrichment of pluripotent progenitor cells can be further accomplished by eliminating the CD34⁺ cells expressing lineage-associated antigens such as CD38 or lacking thy-1.

In addition to the difficulties observed in ex vivo expansion efforts to date, to adequately assay the capability for repopulation and ability to multilineage differentiation of ex vivo cultured HSC an appropriate in vivo model has to be developed. Studies of human stem cell renewal, differentiation and maintenance would be facilitated by the availability of a relevant animal model. In an attempt to develop a relevant and reproducible in vivo transplantation model human hematopoietic cells have been transplanted in immunodeficient mouse strains. A limitation on these mouse strains is that these lack the specific human lymphoid microenvironment to support HSC. Therefor, transplantation of HSCs was generally performed in the presence of high dosages of human cytokines. The development of a humanized murine model by implantation of hematolymphoid tissues into SCID mice (SCID-hu mice) to create a human hematopoietic microenvironment facilitated the development of a useful and relevant in vivo system for assaying the developmental potential of transplantable human HSC. To assay transplantable HSCs with SCID-hu mice similar techniques to those used for secondary transfer and long-term reconstitution of mice can be employed. Such a SCID repopulating cell (SRC) assay has been employed to perform a quantitative assessment of the repopulation capacity of ex vivo cultured cells initiated with CD34⁺CD38⁻ cells. A 4- and 10-fold increase in the number of CD34⁺CD38⁻ cells and CFC respectively were reported in SRC after four days in culture. However after nine days of culture, all SRC were lost despite increases in total cells, CFC counts, and CD34⁺ cells. Therefor, appropriate quantitative assays for transplantable stem cells are essential for the development of culture conditions that support primitive cells.

As described above, others have failed in long term maintenance and expansion ex vivo, culturing CD34⁺ CD38⁻ cells employing conventional methods. Therefor, the need exists to develop an ex vivo expansion method for hematopoietic stem cells. More specifically, the need exists for a method for such ex vivo expansion which can provide HSC which are tranplantable and useful in therapy.

### SUMMARY OF THE INVENTION

A method for the ex vivo expansion of HSC comprises culturing HSC in the presence of a stem cell expansion promoting factor. The expansion promoting factor is obtainable by culturing stromal cells in the presence of sufficient leukemia inhibitory factor to stimulate the cells to produce and secrete the expansion promoting factor. The cultured and expanded HSC retain the capacity for multilineage differentiation and engraftment upon transplantation into patients.

There also is provided a novel stem cell expansion medium which comprises a stem cell expansion promoting factor. The factor can be released from stromal cells upon activation with LIF.

### BRIEF DESCRIPTION OF THE FIGURES

FIG. 1. This figure illustrates the effects of 5 individual cytokines (LIF, Il-3, Il-6, SCF, and GM-CSF) on the proliferative potential of human fetal BM CD34⁺ thy-1⁺ cells in vitro. Data are presented as the total number of hematopoietic cells per well (average of 15 wells) in each culture condition at each weekly time point. The standard deviation for the 15 wells in the LIF-treated cultures at each weekly time point is less than 8% of the mean value.

FIG. 2. This figure illustrates the effects of LIF in combination with other cytokines on the proliferative capacity of freshly purified human fetal BM CD34⁺ thy-1⁺ cells.

FIG. 3. This figure illustrates the kinetics of the proliferative potential of purified human fetal BM CD34⁺ CD38⁻ cells in vitro. The growth factor cocktail included the cytokines Il-3, Il-6, GM-SCF, SCF, and LIF. Data are presented as the total number of hematopoietic cells per well (mean of 15 wells) at each weekly time point. The standard deviation for the 15 wells at each weekly time point is less than 12% of the mean value. For comparison, the kinetic data of CD34⁺ thy-1⁺ cells have been superimposed with the data obtained from CD34+ CD38− cells.

FIG. 4. This figure illustrates hematopoietic reconstitution in the SCID-hu mice with 10,000 ex vivo-expanded CD34⁺ thy-1⁺ cells from 5-week cultures. (A) Intrathymic T-cell development of ex vivo-expanded CD34⁺ thy-1⁺ cells. Graft cells were analyzed by flow cytometry for T-cell markers, CD3, CD4, and CD8, and donor marker (HLA-MA2.1-positive). The percentage of T cells expressing detectable levels of donor-specific HLA class I antigen was recorded. (B) B-cell differentiation and (C) myeloid differentiation of ex vivo-expanded CD34⁺ thy-1⁺ cells in implanted human fetal bone fragment. Graft cells were analyzed for B-cell marker CD19 and myeloid marker CD33, and donor marker HLA-MA2.1.

FIG. 5. This figure illustrates hematopoietic reconstitution in the SCID-hu mice with 10,000 ex vivo-expanded CD34⁺ CD38⁻ cells from 5 week cultures. (A) Intrathymic T-cell development of ex vivo-expanded CD34⁺ CD38⁻ cells. Graft cells were analyzed by flow cytometry for T-cell markers, CD3, CD4, and CD8, and donor marker (HLA-MA2.1-positive). The percentage of T cells expressing detectable levels of donor-specific HLA class I antigen was recorded. (B) B-cell differentiation, and (C) myeloid differentiation of ex vivo-expanded CD34⁺ CD38⁻ cells in implanted human fetal bone fragment. Graft cells were analyzed for B-cell marker CD19 and myeloid marker CD33, and donor marker HLA-MA2.1.

### DETAILED DESCRIPTION OF THE INVENTION

The present invention provides the first ex vivo culture system and process for the maintenance and expansion of hematopoietic stem cells such that said expanded cells can be engrafted into patients without losing their capability for multilineage differentiation. HSC have the capability of both self-renewal and the ability to differentiate into at least eight distinct hematopoietic cell lineages, such as myeloid, B-cell and T-cell lineages. The ex vivo maintenance and expansion of HSC can be achieved by culturing HSC in the presence of a stem cell expansion promoting factor. This factor is obtainable by culturing stromal cells in the presence of leukemia inhibitory factor (LIF). It has been found that following stimulation with LIF, stromal cells produce and secrete a protein product, identified herein as a stem cell expansion promoting factor (SCEPF), which facilitates the maintenance and expansion of hematopoietic stem cells in a culture medium.

In accordance with one embodiment of the present invention, mammalian hematopoietic stem cells (HSC), preferably human HSC, can be expanded ex vivo by culturing isolated HSC in a culture medium which comprises a stem cell expansion promoting factor, said factor obtainable by culturing stromal cells in a culture medium under conditions wherein said stromal cells produce and secrete said expansion promoting factor and then isolating said expansion promoting factor. In an alternative embodiment, stromal cells initially are cultured in a culture medium in the presence of LIF to produce the stem cell expansion promoting factor, the culture medium subsequently is separated from the stromal cells and HSC are cultured in said resultant medium. In a third embodiment of the present invention, a method for the ex vivo maintenance and expansion of HSC comprises culturing isolated HSC in a culture system which comprises a culture medium and stromal cells in the presence of LIF. The HSC are co-cultured with the stromal cells. Such stromal cell culture is pre-established by, for example, seeding 5×10³ to 1×10⁴ stromal cells in 96-well flat bottom plates in 100 μl of long-term culture medium. To this cell stromal cell culture the LIF is added by addition of 100 μl medium providing LIF in a concentration of at least 0.1 ng/ml of medium, preferably in the range of at least about 0.5 ng/ml to 10 ng/ml of medium.

More particularly, in accordance with the first embodiment of this invention, the culture medium comprises any culture medium suitable for culturing hematopoietic stem cells. Such media are known to those of ordinary skill in the art and comprise such components as RPMI 1640, HEPES, FCS, and common antibiotics. The stem cell expansion promotion factor can be obtained by a method which comprises culturing stromal cells in a culture medium to which LIF has been added. Particularly suitable are murine stromal cells. The culturing of the stromal cells is carried out under conditions sufficient to allow the interaction of the LIF with the LIF receptor on the stromal cells such that the cells produce and secrete into the culture medium the stem cell expansion promoting factor. The SCEPF then is isolated from the culture medium and added to any suitable culture medium for the ex vivo maintenance and expansion of hematopoietic stem cells. Such isolation can be accomplished by harvesting the LIF treated stromal cell medium (SCM-LIF), followed by subsequent concentration through size exclusion filtration. To a culture system comprising the medium and stromal cells is added leukemia inhibitory factor (LIF). The LIF can be human LIF or other mammalian LIF, such as murine LIF. Although not wishing to be bound by theory, it appears that the LIF interacts with the LIF receptor on the stromal cells so as to activate the cells. This activation includes a signal transduction response in the cells which induces the production and secretion of one or more stem cell expansion promoting factors or mediators.

Typically the LIF is provided in a concentration of at least about 0.1 ng/ml of medium, preferably at a concentration of at least about 0.5 ng/ml. Typically, the LIF is provided at a concentration in the range of at least about 0.5 ng/ml to at least about 10 ng/ml medium, in particular at a concentration of about 10 ng/ml of medium.

Isolated HSC are cultured in a culture system which comprises a culture medium in the presence of a stem cell expansion promotion factor as described herein. Such a culture system is suitable for achieving a significant expansion, such as a 150-fold expansion, of the HSC. The expanded HSC retain their capability for multilineage differentiation upon introduction into the body of a patient. Desirably, the culture medium for the HSC further comprises at least one cytokine. Preferred cytokines comprise interleukins 3 and 6 (Il-3 and Il-6), stem cell factor (SCF), granulocyte-macrophage colony stimulating factor (GM-CSF), Flt-3 ligand (FL), and thrombopoietin (TPO). A single cytokine can be added or a combination of two or more cytokines can be added to the culture system. Preferably, the medium comprises Il-3 or Il-6, or a combination thereof, or it comprises TPO or CSF or a combination thereof. It has been found that the addition of at least one cytokine can enhance the expansion of HSC by at least about 55%, preferably at least about 120%.

The SCEPF responsible for assisting in the ex vivo expansion of HSC comprises at least one protein having a molecular weight in the range of about 20-30 kD. It has been found that the expansion promoting activity of the stem cell expansion promoting factor is not neutralized by antibodies directed to any of the cytokines listed above which can be present in the stromal cell culture medium following interaction of LIF with the stromal cell LIF receptor. Thus, the SCEPF can be further defined as comprising a protein which is distinct from these cytokines.

In a second embodiment of this invention, HSC can be maintained and expanded ex vivo in the presence of stromal cell medium. In this embodiment, the culture system for the HSC can comprise a culture medium collected from cultured murine stromal cells, the stromal cells having been cultured in the presence of LIF. The culturing of the stromal cells is carried out as described above such that the LIF interacts with the LIF receptor on the stromal cells and the cells produce and secrete into the culture medium the stem cell expansion promoting factor. In this embodiment, the stromal cells then are separated from the culture medium and isolated HSC subsequently are added to the resulting collected culture medium, sometimes referred to as LIF treated stromal cell medium (SCM-LIF). If desired, the (SCM-LIF) can be concentrated prior to use in the ex vivo culture system for the HSC. Such a medium is suitable for achieving a significant expansion, such as a 150-fold expansion, of the HSC. The expanded HSC retain their capability for multilineage differentiation upon introduction into the body of a patient.

In this embodiment, as in the first embodiment, the culture system for the HSC can further comprise at least one additional cytokine. Preferred cytokines comprise those listed above.

In yet a third embodiment of the invention, isolated HSC are added to a culture system comprising the medium, stromal cells and LIF. In such a culture system, HSC were co-cultured on stromal cells in medium for ex-vivo expansion.

It has been found that the presence of LIF in the culture system allows for a 150-fold ex vivo expansion of the HSC in comparison to the expansion of HSC in a comparable culture system in the absence of LIF. It has been found that the ex vivo expansion of HSC in this culture is supported by indirect activation of the HSC by LIF. This is evidenced by the suitability of both human and murine LIF, as murine LIF cannot interact with the human LIF receptor. Murine LIF indirectly stimulates the HSC through co-cultured stromal cells.

As in the preceeding embodiments the ex vivo expansion of the HSC can be further facilitated or enhanced by the addition of at least one cytokine to the culture medium in combination with LIF. Suitable cytokines include those listed above.

In light of the preceding description, one skilled in the art can use the present invention to its fullest extent. The following examples therefor are to be construed as illustrative only and not limiting in relation to the remainder of the disclosure.

### EXAMPLE 1

Preparation of human hematopoietic cells and fluorescence-activated cell sorting.

Human fetal bone, thymus and liver tissues were dissected from 18-24 week old fetuses obtained by elective abortion with approved consent. (Anatomic Gift Foundation, White Oak, Ga.). A sample of each received fetal tissue was stained with a panel of monoclonal antibodies (MoAbs) to HLA to establish the donor allotype. The fetal tissues were used either for construction of SCID-hu mice or for preparation of human HSCs. To purify human HSCs, BM cell suspensions were prepared by flushing split long bones with RPMI 1620 (GIBCO/BRL, Gaithersburg, Md.) containing 2% heat inactivated fetal calf serum (FCS: Gemini Bio-Products, Inc., Calabasas, Calif.). Low density (<1.077 g/ml) mononuclear cells were isolated (Lymphoprep; Nycomed Pharma, Oslo, Norway) and washed twice in staining buffer (SB) consisting of Hanks' Balanced Salt Solution (HBSS) with 2% heat-inactivated FCS and 10 mmol/L HEPES. Samples were than incubated for 10 minutes with 1 mg/ml heat inactivated human gammaglobulin (Gamimmune; Miles Inc., Elkhart, Ind.) to block Fc receptor binding of mouse antibodies. Fluorescein isothiocyanate (FITC)-labeled CD34 MoAbs and phycoerythin (PE)-labeled thy-1 MoAbs (or PE-labeled CD38 MoAbs) were then added at 0.5 to 1 μg/10⁶ cells in 0.1 to 0.3 ml SB for 20 minutes on ice. Control samples were incubated in a cocktail of FITC-labeled and PE-labeled isotype-matched MoAbs. Cells were washed twice in SB, and then resuspended in SB containing 1 μg/ml propidium iodide (Molecular Probes Inc., Eugene, Oreg.) and sorted using the tri-laser fluorescence activated cell sorter MoFlo (Cytomation, Inc., Fort Collins, Colo.). Live cells(ie, those excluding propidium iodide) were always greater than 95%. Sort gates were set based on mean fluoresence intensity of the isotype control sample. Cells were collected in 12- or 24-well plates in RPMI 1640 containing 10% FCS and 10 mmol/l HEPES, counted, and reanalyzed for purity in every experiment. Typically, 450000 to 500000 CD34+ thy-1+ cells were obtained from a single donor. MoAbs for CD34 and CD38 were purchased from Beckton Dickinson (Mountain View, Calif.). MoAbs for thy-1 and isotype controls were purchased from Pharmingen (San Diego, Calif.).

### EXAMPLE 2

In vitro human hematopoietic progenitors/mouse stromal cocultures.

Sorted cells were cultured on a preestablished monolayer of mouse stromal cell line AC6.21. Stromal cells were plated in 96-well-flat-bottom plates 1 week prior in 100 μl of long-term culture medium (LTCM) consisting of RPMI 1640, 0.05 mmol/l 2-mercaptoethanol, 10 mmol/l HEPES, penicillin (50 U/ml), streptomycin (50 mg/ml), 2 mmol/l sodium pyruvate, 2 mmol/l glutamine, and 10% FCS. Twenty CD34⁺ thy-1⁺ cells were distributed in 100 μl of LTCM into each well with preestablished AC6.21 monolayer. The following growth factors, Il-3, Il-6, GM-CSF, SCF, and LIF, were added individually or in combination immediately after seeding the sorted cells at a concentration of 10 ng/ml of each growth factor. Half of the culture medium was replaced weekly with fresh LTCM containing the respective growth factors. The human recombinant Il-3, Il-6, GM-CSF, SCF, and LIF were purchased from R&D Systems (Minneapolis, Minn.).

### EXAMPLE 3

Proliferative analysis, phenotypic analysis and sorting of ex vivo cultured human fetal HSCs.

To determine the extent to which a cytokine or combinations of cytokines support ex vivo expansion of HSCs hematopoietic cells were counted. Cells were harvested without the stromal cells and analyzed for lineage content by flow cytometry by staining with MoAbs for CD19 and CD33 as well as for CD34, thy-1 or CD38. After seven weeks of ex vivo culture all cells were harvested and sorted using flowcytometry. Cells were analyzed by staining with MoAbs for CD19 and CD33 as well as for CD34, thy-1, or CD38. Sorting for HSCs may be obtained by pooling all cells of all 3 populations, either CD19⁺, CD33⁺ and CD34⁺ thy-1⁺ or CD34⁺ CD38⁻ and sorted for either CD34⁺ thy-1+ or CD34⁺ CD38⁻ by flowcytometry.

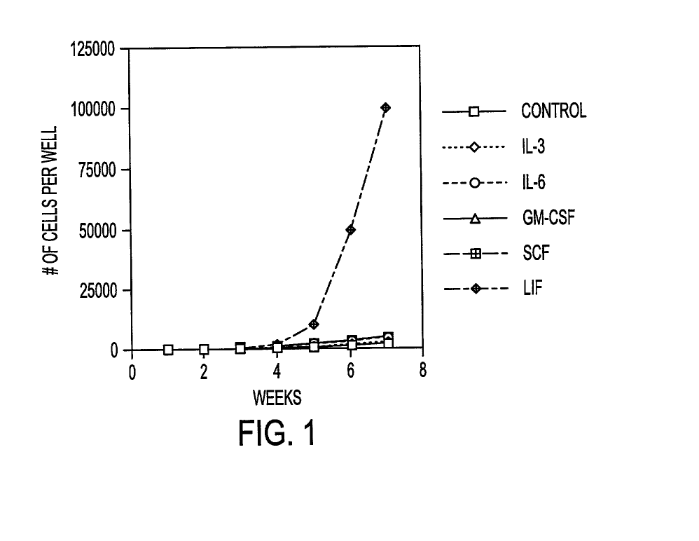

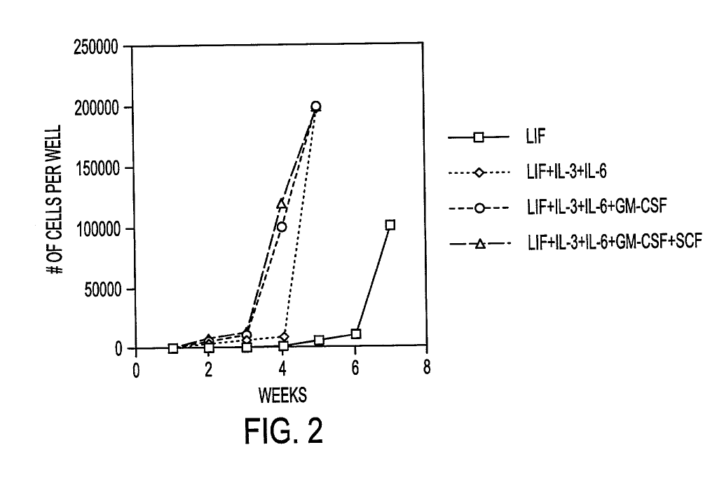

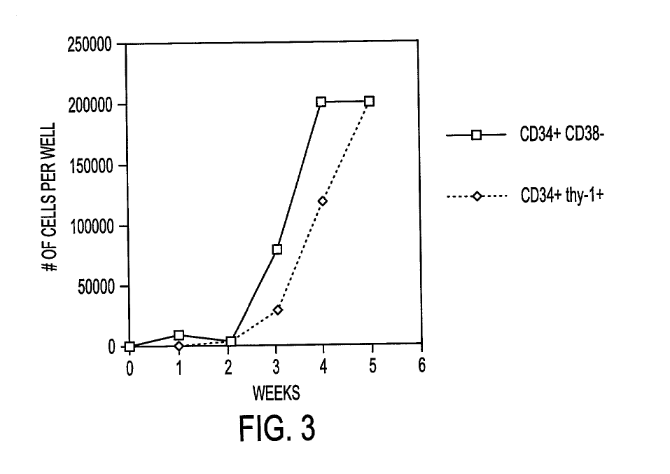

Results show that LIF is the only cytokine that by itself can facilitate proliferation of purified human fetal BM CD34⁺ thy-1⁺ (FIG. 1) or CD34⁺ CD38⁻ cells. In combination with LIF other cytokines such as Il-3, Il-6, GM-SCF, and SCF can establish HSC expansion and accelerate the proliferative kinetics of purified human fetal BM CD34⁺ thy-1⁺ cells (FIG. 2) or CD34⁺ CD38⁻ cells (FIG. 3).

Furthermore, the differentiation potential of purified human fetal BM CD34⁺ thy-1⁺ cells is not dramatically altered as shown in Table I.

TABLE I
Effects of Combinations of Five Cytokines on the
Differentiative Potential of Freshly Purified Human
Fetal BM CD34⁺ thy-1⁺ Cells in Vitro

| | |
|---|---|
| Frequency of | Percentages of |

| | Mixed Lymphoid/ | CD33⁺ | CD19⁺ |
|---|---|---|---|
| Treatments | Myeloid Wells | Cells | Cells |
| Control | 55% (165/300) | 55 ± 5 | 8 ± 2 |
| IL-3 | 51% (152/300) | 45 ± 5 | 10 ± 2 |
| IL-6 | 52% (155/300) | 42 ± 6 | 13 ± 2 |
| GM-CSF | 41% (122/300) | 50 ± 8 | 10 ± 3 |
| SCF | 51% (152/300) | 50 ± 5 | 12 ± 3 |
| LIF | 53% (158/300) | 45 ± 3 | 15 ± 2 |
| LIF + IL-3 + IL-6 | 60% (162/300) | 40 ± 5 | 15 ± 3 |
| LIF + IL-3 + IL-6 + | 58% (173/300) | 55 ± 8 | 13 ± 5 |
| GM-CSF | | | |
| LIF + IL-3 + IL-6 + | 62% (185/300) | 45 ± 3 | 15 ± 2 |
| GM-CSF + SCF | | | |

None of the ex-vivo expanded cell cultures exposed to different cytokines resulted in a significantly different populations of myeloid (CD33⁺) and lymphoid (CD19⁺) cells as compared to the total cell count. Similar results were obtained with freshly purified human fetal CD34⁺ CD38⁻ cells.

The amount of CD34⁺ thy-1⁺ cells in co-culture can be determined as described, and analyzed for its potential for expansion. In LIF treated wells the percentage of CD34⁺ thy-1⁺ cells in positive wells is about 7%. Because each well was initiated with 20 cells and only about 10% of the wells were CD34⁺ thy-1⁺/positive, the expected frequency of cells capable of regenerating CD34+ thy-1+ phenotype is about 1 in 200 within the CD34⁺ thy-1⁺ population. The addition of other human cytokines may facilitate this expansion but cannot support such expansion alone as is shown in Table II.

TABLE II
Effects of Combinations of Five Cytokines on the
Maintenance and Expansion of Freshly Purified
Human Fetal BM CD34⁺ thy-1⁺ Cells in Vitro

| | Frequency of | Percentages |
|---|---|---|
| | CD34⁺ thy-1⁺/ | of CD34⁺ |
| Treatments | Positive Wells | thy-1⁺ cells |
| Control | 0% (0/300) | NA |
| IL-3 | 0% (0/300) | NA |
| IL-6 | 0% (0/300) | NA |
| GM-CSF | 0% (0/300) | NA |
| SCF | 0% (0/300) | NA |
| LIF | 10% (30/300) | 7 ± 1 |
| LIF + IL-3 + IL-6 | 11% (32/300) | 15 ± 2 |
| LIF + IL-3 + IL-6 + | 10% (30/300) | 15 ± 2 |
| GM-CSF | | |
| LIF + IL-3 + IL-6 + | 12% (35/300) | 15 ± 1 |
| GM-CSF + SCF | | |

Considering the total amount of cells per well (200000) and the percentage of CD34⁺ thy-1⁺ cells per positive well is 7%, the total amount of CD34⁺ thy-1⁺ cells per positive well equals approximately 30000. Together with the observation that only 10% to 12% of the wells showed expansion of the CD34+ thy-1+ cells the total expansion of HSC is at least 150 fold under these conditions. Similar results were obtained with freshly purified human fetal CD34⁺ CD38⁻ cells. The total expansion of HSC using CD34⁺ CD38⁻ cells amounted to at least 150 fold under identical conditions.

### EXAMPLE 4

In vivo reconstitution assay in SCID-hu mice.

C.B-17 scid/scid mice were bled under sterile conditions. Mice used for human tissue transplantation were 6 to 8 weeks of age, and the construction of SCID-hu thymus/liver (thy/liv) and bone model mice were constructed as previously described. For thy/liv mice, individual pieces (1 to 2 mm) of human fetal thymus and autologous liver were placed under the kidney capsule of C.B-17 scid/scid mice and allowed to engraft for 3 months before stem cell reconstitution. For bone model mice, pieces of fetal bone were placed subcutaneously and allowed to vascularize for 2 to 3 months. Animals were preconditioned by total body irradiation with 350 rads 4 to 6 hours before they were subjected to stem cell reconstitution. The ability of purified human fetal BM HSCs, including CD34⁺ thy-1⁺ and CD34⁺ CD38⁻ populations, either fresh uncultured or ex-vivo expanded, to reconstitute thymus and BM was tested by indirect inoculation into irradiated grafts (thy/liv and bone, the graft is always selected to be HLA-MA2.1-negative). A limiting dilution experiment was conducted to determine quantitatively the transplantable cells in the freshly purified human fetal BM CD34⁺ thy-1⁺ population.

For a typical donor reconstitution derived from freshly purified CD34+ thy-1+ cells were evident in 87%, 20%, 7% and 0% of the bone crafts and 93%, 20%, 7%, and 0% of the thy/liv crafts when transplantation was performed with 10000, 3000, 1000, and 300 cells respectively. The percentage of donor derived cells in the bone grafts of reconstituted animals was 41%±10%, 9%±3%, 2.2% from an injected cell dose of 10000, 3000 and 1000 respectively. The percentage of donor derived cells in the thymic grafts of reconstituted animals was 50%±8%, 12%±4%, and 3.2% from an injected cell dose of 10000, 3000 and 1000 respectively. For other reconstitution experiments 10000 cells were used because 10000 CD34+ thy-1+ cells purified from fresh fetal BM reproducibly establish long-term hematopoietic reconstitution in greater than 90% of SCID-hu mice. Engraftment was analyzed at 3 to 4 months postinjection. Human bones were removed and split open to flush the marrow cavity with SB. Collected cells were spun down and the pellet was resuspended for 5 minutes in a red blood cell lysing solution. Cells were washed twice in SB and counted before being stained for 2-color immunofluorescence with directly labeled MoAbs against HLA allotypes in combination with CD19 and CD33. Human thymus grafts were recovered, reduced to cellular suspension, and subjected to 2-color immunofluorescence analysis using directly labeled MoAbs against HLA allotypes in combination with CD3, CD4 and CD8. Cells were analyzed on a FACScan fluorescent cell analyzer. FITC- or PE-labeled CD19, CD33, CD3, CD4 and CD8 were purchased from Pharmingen (San Diego, Calif.).

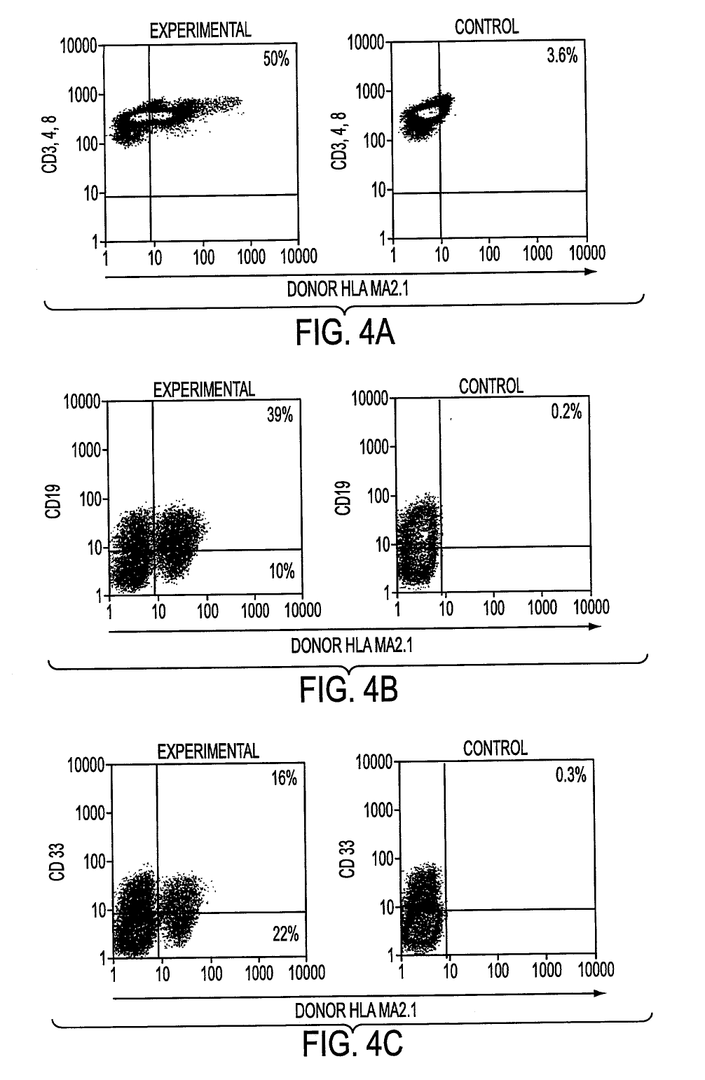

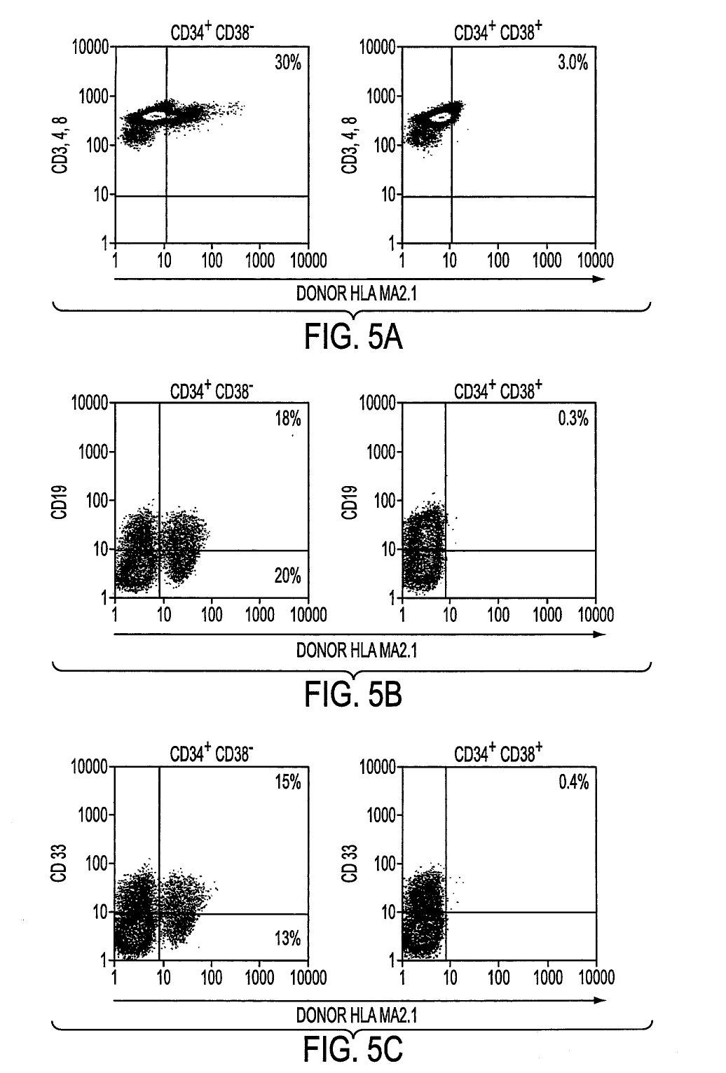

The expanded HSC so engrafted in the SCID-hu mice show multilineage differentiation (FIG. 4). Transplantation with 10000 ex vivo expanded cells shows that the engrafted human thymus contained 50% ex vivo expanded CD34+ thy-1⁺ derived thymocytes. These cells were further analyzed with T-cell markers CD3, CD4, and CD8 and showed a normal T-cell maturation pattern. The engrafted human bone fragment of this SCID-hu mouse contained 39% donor-derived CD19⁺ B cells and 16% donor-derived CD33⁺ myeloid cells. Also the ex vivo expanded HSCs gave rise to almost identical reconstitution rates in both the thy/liv and bone mice from 10000 cells as compared to 10000 cells from freshly purified human fetal BM. Similar results were obtained when ex vivo expanded purified human fetal CD34⁺ CD38⁻ cells were so engrafted in SCID-hu mice (FIG. 5).

### EXAMPLE 5

Preparation of stromal-conditioned media from untreated (SCM) and LIF treated stromal cell cultures (SCM-LIF).

Stromal-conditioned medium were harvested from a confluent layer of mouse stromal cell line AC6.21. Stromal cells were cultured in long-term culture medium (LTCM) consisting of RPMI 1640, 0.05 mmol/l 2-mercaptoethanol, 10 mmol/l HEPES, penicillin (50 U/ml), streptomycin (50 mg/ml), 2 mmol/l sodium pyruvate, 2 mmol/l glutamine and 10% FCS at 37° C. in a humidified atmosphere with 5% CO2. A complete medium change was made with fresh LTCM containing 10 ng/ml LIF when the stromal cell layer was confluent. Conditioned medium from stromal cells was harvested every 3 days by replacing half of such media with fresh LTCM containing 10 ng/ml LIF for a period of up to four weeks. The SCM-LIF was centrifuged at 1300 rpm for 10 minutes to remove nonadherent cells and filtered through a 0.45-μm pore filter with low protein binding (Sterivex-HV; Millipore, Bedford, Mass.). To concentrate SCM-LIF crude supernatants were first concentrated with a DC10 concentrator using a 100 kD molecular weight cutoff hollow-fiber cartridge (Amicon Inc, Danvers, Mass.). The concentrate was then clarified by filtering with a 5 kD molecular weight cutoff cartridge. With such concentration SCM-LIF was concentrated 40-fold. SCM can be obtained similarly by culturing the stromal cells in the absence of LIF and harvesting the conditioned media the same.

The SCM-LIF was fractionated by molecular weight by using similar hollow-fiber cartridges (Amicon Inc, Danvers, Mass.) in a concentrator as described above, each with a different molecular weight cutoff. In each concentrator 10 ml of SCM-LIF or a fractionated sample thereof was spun in a centrifuge at 3500×G for a period of time sufficient to establish a 10 fold reduction in the volume for the retained concentrate. Following centrifugation of the concentrator both the flow-thru and retained concentrate fractions were collected from each filtration with a hollow-fiber cartridge of a particular molecular weight cutoff. The flow-thru of such size exclusion filtration may have been further submitted for a second round of filtration in a concentrator in which the holow-fiber cartridge has a different molecular weight cutoff. The fractions so obtained were used in a culture system comprising medium and HSC as taught in example 6 to determine the fraction containing the SCEPF activity to expand HSC. The fraction comprising proteins in the range of 8 kD to 30 kD retained the activity for SCEPF. The results are shown in Table III in which the concentration of the proteins in each fraction is normalized to an equivalent of 1×SCM-LIF.

TABLE III
SCEPF activity in the 8-30 kD fraction
from size-exclusion filtration.

| | Frequency of | Percentages |
|---|---|---|
| Culture | CD34⁺ thy-1⁺/ | of CD34⁺ |
| Conditions | Positive Wells | thy-1⁺ Cells |
| Positive control | 100% (10/10) | 8 ± 2 |
| Negative control | 0% (0/10) | NA |
| <8 kD | 0% (0/10) | NA |
| 8-30 kD | 70% (7/10) | 11 ± 4 |
| 30-50 kD | 0% (0/10) | NA |
| 50-100 kD | 0% (0/10) | NA |
| >100 kD | 0% (0/10) | NA |

Further, a fraction containing proteins in the 8-30 kD range, obtained through a method as described above, was subjected to additional fractionation in the same manner using concentrators with hollow-fiber tube cartridges of different molecular weight cutoffs. These fractions were used in a culture system comprising medium and HSC as taught in example 6 to determine which fraction had retained the ability to expand HSC. A fraction so obtained comprising proteins between 20-30 kD was the only fraction showing HSC expansion activity, thus comprising the SCEPF protein.

### EXAMPLE 6

SCM-based HSC expansion culture system.

Culture media containing 5%, 10% and 25% SCM-LIF are prepared by mixing fresh LTCM with appropriate amounts of unconcentrated SCM-LIF. Culture media containing 50%, 100%, 200% and 400% SCM-LIF may be obtained by mixing fresh LTCM with respective amounts of concentrated SCM-LIF. Freshly purified CD34+ thy-1+ cells may be cultured in LTCM containing 10 ng/ml of Il-1, IL-6, GM-CSF, SCF, and different concentrations of SCM-LIF. A complete media exchange is made every 3 days and replaced with LTCM containing desired cytokines and amounts of SCM-LIF.

### EXAMPLE 7

Effect of SCM-LIF on ex vivo proliferation and differentiation of human fetal BM CD34⁺ thy-1⁺ cells.

In comparison with CD34⁺ thy-1 cells in a co-culture system with stromal AC6.21 cells in the presence of LIF as positive control and the same culture system in the absence of LIF as negative control, CD34⁺ thy-1⁺ cells cultured in different concentrations of SCM-LIF as taught in example 6 showed increasing frequency of positive wells as is shown in Table IV.

TABLE IV
Effects of SCM-LIF on the maintenance and
expansion of freshly purified human fetal BM
CD34⁺ thy-1⁺ cells in vitro

| | Frequency of | Percentage |
|---|---|---|
| Culture | CD34⁺ thy-1⁺ | of CD34⁻ |
| Conditions | -Positive Wells | thy-1⁻ Cells |
| Positive Control | 100% (10/10) | 8 ± 2 |
| Negative Control | 0% (0/10) | N/A |
| 0% SCM-LIF | 0% (0/10) | N/A |
| 5% SCM-LIF | 0% (0/10) | N/A |
| 10% SCM-LIF | 10% (1/10) | 3.6 |
| 25% SCM-LIF | 40% (4/10) | 5 ± 3 |
| 50% SCM-LIF | 70% (7/10) | 10 ± 4 |
| 100% SCM-LIF | 100% (10/10) | 14 ± 4 |
| 200% SCM-LIF | 100% (10/10) | 18 ± 4 |
| 400% SCM-LIF | 100% (10/10) | 18 ± 3 |

Also, at 100% SCM-LIF the frequency of positive wells is identical to the positive control. In contrast, as is shown in Table V, for CD34⁺ thy-1⁺ cells cultured in SCM only no ex vivo expansion could be detected, even in the presence of LIF.

TABLE V
SCM-LIF maintains its activity to facilitate ex
vivo expansion of freshly purified human fetal BM
CD34⁺ thy-1⁺ cells in the presence of SCM

| | Frequency of | Percentage |
|---|---|---|
| Culture | CD34⁺ thy-1⁺ | of CD34⁻ |
| Conditions | -Positive Wells | thy-1⁻ Cells |
| Positive control | 100% (10/10) | 9 ± 3 |
| Negative control | 0% (0/10) | N/A |
| 200% SCM | 0% (0/10) | N/A |
| 400% SCM | 0% (0/10) | N/A |
| 200% SCM-LIF | 100% (10/10) | 17 ± 3 |
| 200% SCM-LIF + | 100% (10/10) | 17 ± 4 |
| 200% SCM | | |
| 200% SCM-LIF + | 100% (10/10) | 18 ± 6 |
| 400% SCM | | |

The differentiation potential of purified in a SCM-based culture system as analyzed by flowcytometry for the presence of CD19+ lymphoid cells and CD33+ myeloid cells showed that regardless of different treatment, varying the concentrations of SCM-LIF, both CD19⁺ and CD33⁺ cells were generated at similar levels (about 50% of the wells). Therefore, SCM-LIF is capable of providing a suitable environment for multipotential CD34⁺ thy-1⁺ cells to differentiate into both B cells and myeloid cells similar to the stromal-based culture system as well as the ex vivo expansion of CD34⁺ thy-1⁺ cells.

### EXAMPLE 8

Enhancement of the proportion of CD34⁺ thy-1⁺ cells in cultures with SCM-LIF in the presence of several combinations of cytokines.

The activity of SCM-LIF to support an ex viva culture system for expansion op HSCs may be attributed to a SCEPF (stem cell expansion promoting factor). The SCEPF does not comprise any of the prominent stem cell cytokines since neutralizing antibodies cannot block the ex vivo stem cell expansion. When CD34⁺ thy-1⁺ cells were cultured in 200% SCM-LIF in the presence of 0.1 to 10 μg/ml of neutralizing antibody against each of the cytokines from the group of GM-CSF, SCF, Il-3, Il-6, FL, and TPO the ex vivo stem cell expansion was not affected. Furthermore, culturing of the CD34⁺ thy-1⁺ cells in 200% SCM in the presence of 10 ng/ml of LIF and 10 ng/ml of each of those cytokines either alone or in combination does not result in ex vivo expansion of HSCs. However, when 10 ng/ml of the 6 cytokines either alone or in combination were supplemented to the SCM-LIF based culture system with 200% SCM-LIF these cytokines were capable of further enhancing the proportion of CD34⁺ thy-1⁺ cells similar as observed in the stromal-based culture system, see Table VI.

TABLE VI
Conditions that significantly enhance the
proportion of cells with CD34⁺ thy-1⁺
phenotype in the cultures

| | Frequency of | Percentage |
|---|---|---|
| | CD34⁺ thy-1⁺⁻ | of CD34⁻ |
| Treatments | Positive Wells | thy-1⁺ Cells |
| None | 100% (20/20) | 9 ± 2 |
| IL-3 + IL-6 + SCF | 100% (20/20) | 14 ± 2 |
| IL-3 + IL-6 + SCF + FL | 100% (20/20) | 17 ± 3 |
| GM-CSF + IL-3 + IL-6 + SCF | 100% (20/20) | 18 ± 4 |
| GM-CSF + IL-3 − IL-6 − SCF + FL | 100% (20/20) | 18 ± 4 |
| TPO + SCF | 100% (20/20) | 14 ± 2 |
| TPO + FL + SCF + IL-3 | 100% (20/20) | 16 ± 2 |
| TPO + FL − SCF + IL-6 | 100% (20/20) | 18 ± 3 |
| TPO + FL + SCF + IL-3 + IL-6 | 100% (20/20) | 20 ± 4 |

What is claimed is:

1. A method for the ex-vivo maintenance and expansion of hematopoietic stem cells (HSC) which comprises culturing HSC in a culture medium comprising a stem cell expansion promoting factor, wherein said expansion promoting factor is obtainable by a method which comprises:
culturing stromal cells in the presence of sufficient LIF to stimulate said cells to produce and secrete said expansion promoting factor.

2. A method according to claim 1, wherein the HSC retain their ability for multilineage differentiation.

3. A method according to claim 1, wherein the HSC retain their ability to differentiate to a myeloid lineage.

4. A method according to claim 1, wherein the HSC retain their ability to differentiate to a B-cell lineage.

5. A method according to claim 1, wherein the HSC retain their ability to differentiate to a T-cell lineage.

6. A method according to claim 1, wherein the HSC are human hematopoietic stem cells.

7. A method according to claim 1, wherein said stromal cells comprise murine stromal cells.

8. A method according to claim 1, wherein said LIF is provided in a minimum concentration of at least about 0.5 ng/ml of medium per 1×10⁴ stromal cells per 100 μl medium.

9. A method according to claim 1, wherein said culture medium further comprises an additional mammalian cytokine.

10. A method according to claim 6, wherein the cytokine comprises Il-3, Il-6, SCF, GM-CSF, FL, thrombopoietin (TPO) or a combination thereof.

11. A method according to claim 10, wherein said culture medium further comprises Il-3 or Il-6.

12. A method according to claim 10, wherein said culture medium further comprises TPO or SCF.

13. A method according to claim 1, wherein said stem cell expansion promoting factor has a molecular weight in the range of about 20-30 kD.

14. A method for the ex vivo maintenance and expansion of hematopoietic stem cells (HSC) which comprises:
(a) adding a culture medium to stromal cells and culturing said stromal cells in the presence of an amount of LIF sufficient to activate LIF receptors on said stromal cells;
(b) separating said stromal cells from said culture medium;
(c) adding isolated HSC to said medium; and
(d) culturing said HSC in said medium.

15. A method for the ex-vivo expansion of mammalian hematopoietic stem cells (HSC) which comprises culturing isolated HSC in a culture system comprising a culture medium and stromal cells in the presence of an amount of leukemia inhibitory factor (LIF) sufficient to activate LIF receptors on said stromal cells and thereby stimulate the production and secretion of a stem cell expansion factor.

16. A method according to claim 14 or 15, wherein said stromal cells comprise murine stromal cells.

17. A method according to claim 14 or 15, wherein said medium in which said HSC are cultured further comprises an additional cytokine.

18. A method according to claim 17, wherein said cytokine comprises Il-3, Il-6, SCF, GM-CSF, FL, thrombopoietin (TPO), or a combination thereof.

19. A culture system suitable for the ex-vivo expansion of hematopoietic stem cells (HSC) which comprises a culture medium and stem cell expansion promoting factor.

20. A culture system according to claim 19, which further comprises a cytokine.

21. A culture system according to claim 20, wherein said cytokine comprises Il-3, Il-6, SCF, GM-CSF, FL, thrombopoietin (TPO) or a combination thereof.

22. A culture system according to claim 21, which comprises Il-3 or Il-6.

23. A culture system according to claim 21, which comprises TPO or CSF.

24. A culture system according to claim 19, wherein said stem cell expansion promoting factor is obtainable by a method which comprises adding a culture medium to stromal cells, adding LIF to said medium, and culturing said stromal cells such that they secrete said stem cell expansion promoting factor.

25. A culture system according to claim 23, wherein said stem cell expansion promoting factor has a molecular weight in the range of about 20-30 kD.

26. A hematopoietic stem cell expansion factor, said factor having a molecular weight in the range of about 20-30 kD and obtainable by a method which comprises culturing stromal cells in the presence of sufficient LIF to stimulate said cells to produce and secrete said stem cell expansion factor.

27. A hematopoietic stem cell expansion factor according to claim 26, wherein said stromal cells comprise murine stromal cells.
